(19) United States
(12) Patent Application Publication
(10) Pub. No.: US 2008/0274075 A1
(43) Pub. Date: Nov. 6, 2008

(54) Poly(Ethylene Glycol) Derivatives and Process For Their Coupling to Proteins

(75) Inventor: Florencio Zaragoza Dorwald, Smorum (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk Healthcare A/G, Zurich (CH)

(21) Appl. No.: 11/914,850

(22) PCT Filed: May 19, 2006
(86) PCT No.: PCT/EP06/62464

§ 371 Date: May 30, 2008

(30) Foreign Application Priority Data
May 20, 2005 (EP) 05104309.9

(51) Int. Cl. A61K 31/765; C08G 73/00; A61P 5/00; C08L 89/00

(52) U.S. Cl. 424/78.17; 528/367; 525/54.1

(57) ABSTRACT

Novel compounds, including PEGylated proteins of the formula, methods for preparing such compounds, methods of using such compounds, and other compositions and methods, are provided.

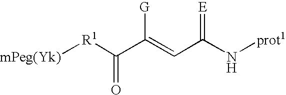

### FIELD OF THE INVENTION

The present invention relates to new derivatives of poly(ethylene glycol) and to methods for the covalent attachment of poly(ethylene glycol) to proteins.

### BACKGROUND OF THE INVENTION

The covalent attachment of poly(ethylene glycol) (PEG) to peptides and proteins with the aim of obtaining analogues with improved pharmacological properties is a well-established strategy (Zobel et al., Bioorg. Med. Chem. Lett. 2003, 13, 1513-1515). In particular derivatives of high-molecular-weight PEG, e.g. mPEG(40k), are useful, because these can usually not be cleared by renal filtration, and thus have prolonged half-lives in plasma.

Covalent attachment of compounds to proteins is generally performed by acylation (amide-bond formation with lysine side-chains or with the N-terminal amino acid) or by a condensation reaction of a suitable alkoxylamine, hydrazine, or 2-aminothiol with a protein-derived ketone or aldehyde to yield an oxime, a hydrazone, or a thiazolidine, respectively (Shao and Tam, J. Am. Chem. Soc. 1994, 117, 3893-3899). These reactions can only be conducted under conditions where the protein and the derivatizing reagent are dissolved, and the identification of such reaction conditions may require a long and tedious adjustment of critical parameters, such as solvent, pH, concentration, additives, and temperature.

The rate of formation of oximes from alkoxylamines and carbonyl compounds such as aldehydes or ketones is highly pH-dependent, and usually proceeds fastest under slightly acidic conditions (pH 2-4). Acceptable reaction rates may be attained at pH 1-6. Related condensation reactions, such as the formation of hydrazones, thiazolidines, or 1,3-thiazines show a similar pH-dependency. Proteins which contain numerous acidic amino acids have a low solubility under acidic conditions (pH<7), and such proteins are difficult to condense with PEG using oxime-formation or related, known reactions. For instance, the isoelectric point of human growth hormone (hGH), i.e. the pH at which its solubility in water is lowest, is 5.1, and if an oximation of a hGH-derived aldehyde or ketone is attempted at pH 4, precipitation of the protein usually occurs, with a resulting poor yield. The precipitation will be further promoted by the presence of PEG, because PEG has a high affinity for water and induces the precipitation of proteins. Furthermore, the PEGylation of large proteins with high molecular weight PEG (e.g. PEG(20k), PEG(30k), PEG (40k), PEG(60k), etc) usually requires high concentrations of reactants in order to proceed sufficiently quickly. Such high concentrations of reactants will further promote the precipitation of acidic proteins.

Hence, the identification of new coupling reactions, which can be conducted under basic reaction conditions, will be of high general interest. Such methods would enable the PEGylation of acidic proteins, with a much lower risk of precipitation. Among other things, the invention provides such methods. These and other useful features, aspects, and advantages of the invention will be apparent to ordinarily skilled artisans from the disclosure provided herein.

### SUMMARY OF THE INVENTION

The present inventor has surprisingly found that PEG-derived cyanoacetamides and PEG-derived phosphonium salts undergo smooth reaction with protein-derived aldehydes or ketones in water under basic reaction conditions. From this discovery, a number of inventive compositions and methods have been conceived.

In one exemplary embodiment, the invention relates to a compound of formula I

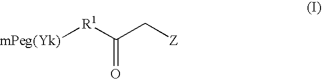

wherein
Y represents an integer from 1 to 140 (as indicated elsewhere, “k” indicates that the variable Y actually refers to a multiple of 1000);
R¹ represents a diradical selected from

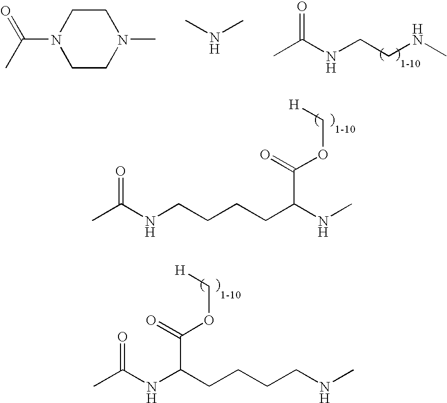

and
Z represents an electron-withdrawing group;

In one embodiment, the invention relates to a method of covalently attaching a PEG derivative to a protein, the method comprising treating an aldehyde or ketone derivative of said protein with a compound of formula I at basic pH.

In one embodiment, the invention relates to PEGylated proteins obtained by the reaction of the present invention. In particular, the invention relates to a compound of formula II

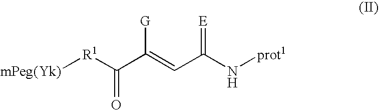

wherein Y is an integer from 1 to 140;
R¹ represents a diradical selected from

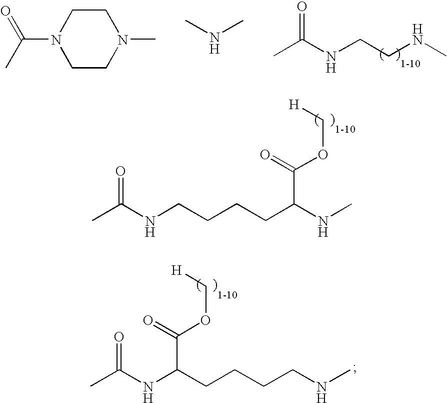

G represents an electron-withdrawing group or hydrogen;
E represents O or H₂;
and -prot¹ represents a radical derived from a protein by formal removal of either the N-terminal amino group or the hydroxyl group of a glutamic acid side chain.

In one embodiment, the invention relates to a compound of formula III

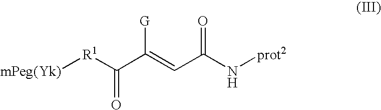

wherein Y is an integer from 1 to 140;
R¹ represents a diradical selected from

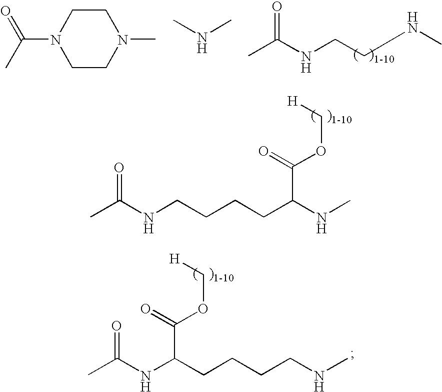

G represents an electron-withdrawing group or hydrogen;
E represents O or H₂;
and -prot² represents a radical derived from a protein by formal removal of the N-terminal amino group.

In one embodiment, the invention relates to a compound of formula IV

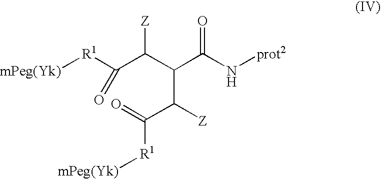

wherein Y is an integer from 1 to 140;
R¹ represents a diradical selected from

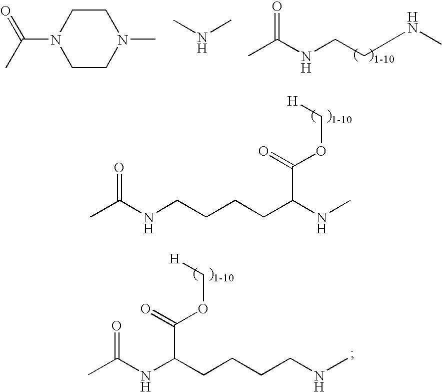

Z represents an electron-withdrawing group; and
prot² represents a radical derived from a protein by formal removal of the N-terminal amino group.

In one embodiment, the invention provides compounds of formula II, III or IV for use in therapy.

In one embodiment, the invention relates to a composition comprising a compound of formula II, III or IV, and in particular a pharmaceutical composition.

In one embodiment, the invention relates to a method of treating diseases, the method comprising administering a therapeutically effective amount of a compound of formula II, III or IV to a patient in need thereof.

In one embodiment, the invention relates to the use of a compound of formula II, III or IV in the manufacture of a medicament.

In one embodiment, the invention provides a method for improving pharmacological properties of a protein, the method comprising contacting said protein with a compound of formula I at basic pH.

These and other useful aspects, features, and benefits of the invention are further described elsewhere herein.

The term “unnatural amino acid” refers to any compound comprising at least one primary or secondary amino group and at least one carboxyl group, without being alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The term “human growth hormone” refers to a protein with the following amino acid sequence:

FPTIPLSRLFDNAMLRAHRLHQLAFDTYQEFEEAYIPKEQKYSFLQNPQT
SLCFSESIPTPSNREETQQKSNLELLRISLLLIQSWLEPVQFLRSVFANS
LVYGASDSNVYDLLKDLEEGIQTLMGRLEDGSPRTGQIFKQTYSKFDTNS
HNDDALLKNYGLLYCFRKDMDKVETFLRIVQCRSVEGSCGF.

The term “halogen” includes F, Cl, Br and I.

In the present context, the term “alkyl” is intended to indicate a straight, branched and/or cyclic saturated monovalent hydrocarbon radical having from one to ten carbon atoms, also denoted as C₁₋₁₀-alkyl. In particular, alkyl is intended to indicate C₁₋₆-alkyl, such as e.g. methyl, ethyl, n-propyl, isopropyl, n-butyl, sec-butyl, isobutyl, tert-butyl, n-pentyl, 2-methylbutyl, 3-methylbutyl, 4-methylpentyl, n-hexyl, 1,1-dimethylpropyl, 1,2-dimethylpropyl, 2,2-dimethylpropyl (neopentyl) and 1,2,2-trimethylpropyl.

The term “aryl” as used herein is intended to include carbocyclic aromatic ring systems such as phenyl, biphenylyl, naphthyl, anthracenyl, phenanthrenyl, fluorenyl, indenyl, pentalenyl, azulenyl and the like. Aryl is also intended to include the partially hydrogenated derivatives of the carbocyclic systems enumerated above. Non-limiting examples of such partially hydrogenated derivatives are 1,2,3,4-tetrahydronaphthyl, 1,4-dihydronaphthyl and the like.

The term “optionally substituted” as used herein means that the groups in question are either unsubstituted or substituted with one or more of the substituents specified. When the groups in question are substituted with more than one substituent, the substituents may be the same or different.

The term “PEG” is intended to indicate polyethylene glycol of a molecular weight between approximately 100 and approximately 1,000,000 Da, including analogues thereof, wherein for instance the terminal OH-group has been replaced by an alkoxy group, such as e.g. a methoxy group, an ethoxy group or a propoxy group. In particular, the PEG wherein the terminal —OH group has been replaced by methoxy is useful, and it is being referred to as mPEG.

The term “mPEG” (or more properly “mPEGyl”) means a polydisperse or monodisperse radical of the structure

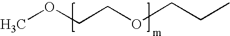

wherein m is an integer larger than 1. Thus, a mPEG wherein m is 90 has a molecular weight of 3991 Da, i.e. approx 4 kDa. Likewise, a mPEG with an average molecular weight of 20 kDa has an average m of 454. Due to the process for producing mPEG these molecules often have a distribution of molecular weights. This distribution is described by the polydispersity index.

The term “polydispersity index” as used herein means the ratio between the weight average molecular weight and the number average molecular weight, as known in the art of polymer chemistry (see e.g. “Polymer Synthesis and Characterization”, J. A. Nairn, University of Utah, 2003). The polydispersity index is a number which is greater than or equal to one, and it may be estimated from Gel Permeation Chromatographic data. When the polydispersity index is 1, the product is monodisperse and is thus made up of compounds with a single molecular weight. When the polydispersity index is greater than 1 it is a measure of the polydispersity of that polymer, i.e. how broad the distribution of polymers with different molecular weights is.

The use of, for example, “mPEG(20k)” in formulas, compound names or in molecular structures indicates an mPEG residue wherein mPEG is polydisperse and has a molecular weight of around 20 kDa.

The polydispersity index typically increases with the molecular weight of the PEG or mPEG. When reference is made to 5 kDa PEG and in particular 5 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 10 kDa PEG and in particular 10 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 15 kDa PEG and in particular 15 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 20 kDa PEG and in particular 20 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 30 kDa PEG and in particular 30 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 40 kDa PEG and in particular 40 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 60 kDa PEG and in particular 60 kDa mPEG it is intended to indicate a compound (or in fact a mixture of compounds) with a polydisperisty index below 1.06, such as below 1.05, such as below 1.04, such as below 1.03, such as between 1.02 and 1.03.

The term protein is intended to indicate a compound comprising two or more amino acids bonded via a peptide bond, e.g., peptides and polypeptides. Typically, a protein comprises 30 or more, such as 50 or more, such as 100 or more amino acid residues. The term is also intended to include polypeptides further natural or un-natural derivatisation, such as e.g. glycosylation, attachment of PEG or lipophilic groups, and polypeptides including further groups, such as e.g. prosthetic groups, such as e.g. heme. The term is also intended to include higher order structures, such as dimers and multiple chain proteins.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on e.g. the severity of the disease or injury as well as the weight, sex, age and general state of the subject. It will be understood that determining an appropriate dosage may be achieved using routine experimentation, by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs. Nonetheless, it should be recognized that therapeutic regimens and prophylactic (preventative) regimens represent separate aspects of the invention.

### DETAILED DESCRIPTION OF THE INVENTION

In one embodiment, the invention relates to compounds of the formula I

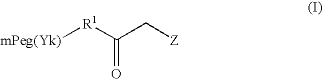

In one embodiment, Z represents electron-withdrawing groups, such as e.g. cyano, nitro, P(R²)₃⁺X⁻, —S(═O)R³, —S(═O)₂R⁴, or —C(═O)R⁵, wherein X represents halogen, BF₄, or PF₆;

R², R³, R⁴, and R⁵ independently represent aryl, optionally substituted with halogen, C₁₋₆-alkyl, cyano, or carboxyl; C₁₋₆-alkyl, optionally substituted with cyano; or NR⁶R⁷, wherein R⁶ and R⁷ independently represent hydrogen or C₁₋₆-alkyl. In particular, Z represents cyano or —PPh₃Cl, wherein Ph represent phenyl.

In one embodiment, R¹ represents

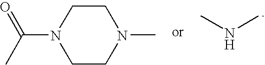

In one embodiment, Y represents 10, 20, 30, 40, or 60.

Particular examples of compounds of formula I include:

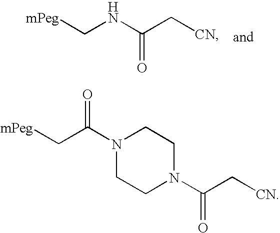

In particular, compounds of formula I include
(1-(4-(4-(1,3-bis(mPEG(20K)oxy)-2-propyloxy)butyryl)piperazin-1-yl)acet-2-yl)triphenyl-phosphonium chloride; and
N-(mPEG(20k)yl)cyanoacetamide.

PEG-derived cyanoacetamides can be prepared by acylation of a PEG-derived amine with cyanoacetic acid, or by acylation of a mono-cyanoacetyl diamine with a PEG-derived acylating reagent, for instance an N-hydroxysuccinimidyl ester, as sketched below:

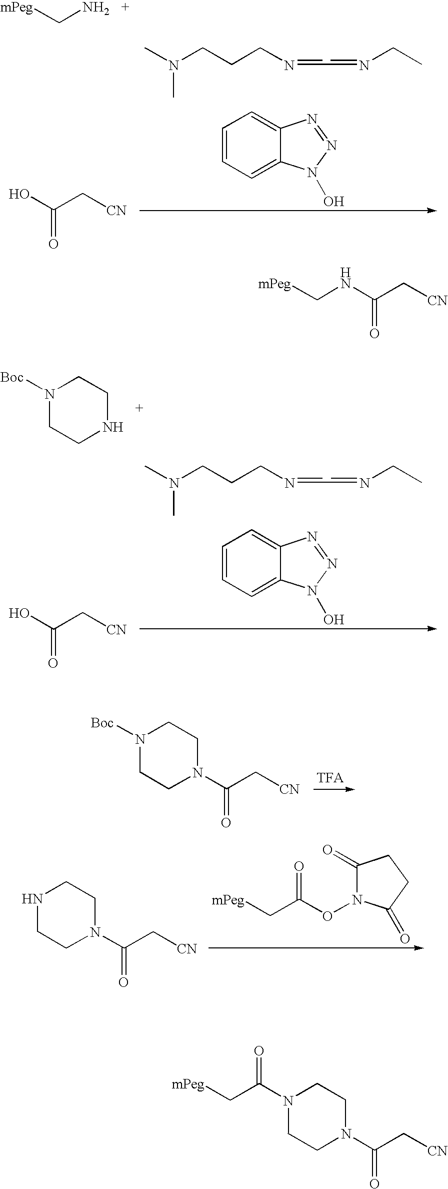

Treatment of a protein-derived aldehyde or ketone with these reagents in water or aqueous solution at basic pH, such as pH 8-12, preferentially at pH 9-11, yields the PEGylated protein. Depending on the ratio of PEG-derived cyanoacetamide and protein-derived aldehyde or ketone, a single or a twofold PEGylation of the protein will occur. This is because the initial product of condensation is an electron-poor alkene, which readily undergoes Michael addition with carbon nucleophiles, as sketched below for a specific example of protein-derived aldehyde:

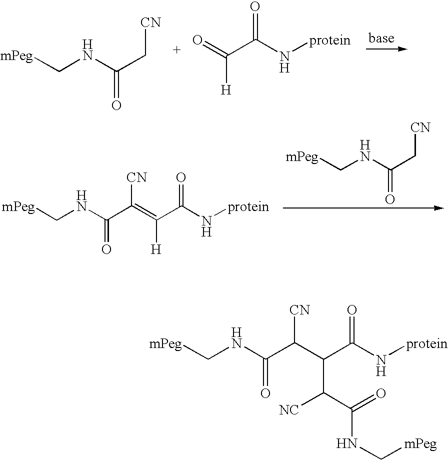

Thus, this method enables a single or two-fold PEGylation of proteins, in particular acidic proteins, and thus the preparation of derivatives of high-molecular weight PEG, such as mPEG(40k), mPEG(60k), mPEG(80k), or mPEG(120k). Such compounds will usually no longer be eliminated by renal filtration, and should therefore have long plasma-half-lives in vivo.

PEG-derived phosphonium salts can be prepared by acylation of a mono-phosphonioacetyl diamine with a PEG-derived acylating reagents, for instance an N-hydroxysuccinimidyl ester, as sketched below:

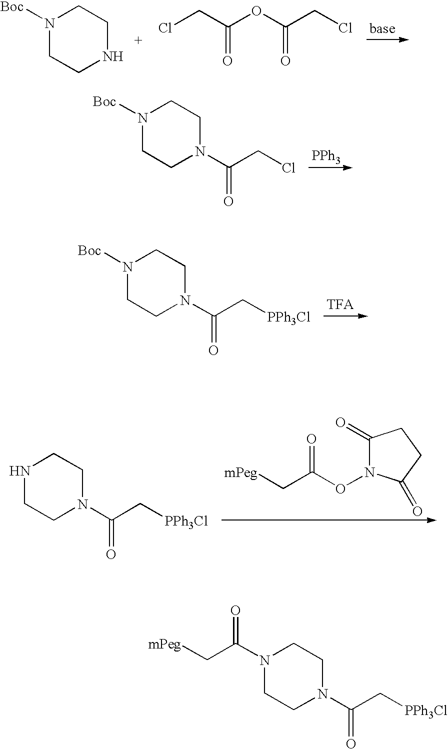

Treatment of a protein-derived aldehyde or ketone with these reagents in water or aqueous solution at basic pH, such as pH 8-12, preferentially at pH 9-11, yields the PEGylated protein by Wittig reaction, as sketched below for a specific example of protein-derived aldehyde:

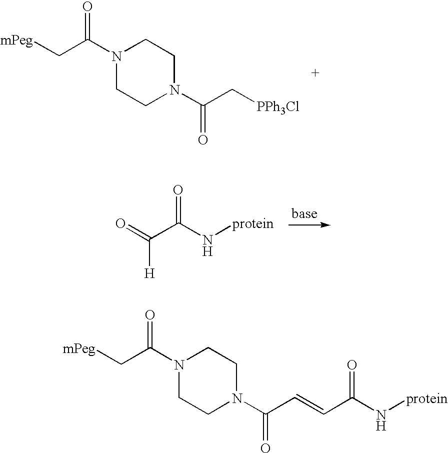

Protein-derived aldehydes or ketones may be prepared by several routes. The present invention applies to any protein-derived aldehyde or ketone, irrespective of the way in which it was prepared.

One possibility for preparing protein-derived aldehydes is a periodate-mediated oxidation of a protein, which contains serine or threonine as N-terminal amino acid. This may either be a protein which already contains such N-terminal amino acid, or it may be an analogue of a protein, to which an N-terminal serine or threonine has been attached. Such analogues may be prepared by standard genetic techniques. Alternatively, additional amino acids may be attached to the N-terminal of a protein with the aid of an enzyme, e.g. an aminopeptidase, in the presence of a large excess of an amino acid. The elongated analogue may be an analogue to which only one serine has been added to the protein. Alternatively, an analogue may be prepared which contains several amino acids between the N-terminal of the original protein and added serine, i.e. to obtain a compound of the general formula Ser-XX-protein, wherein XX represents any sequence of 1-50 natural and/or un-natural amino acids.

Alternatively, a protein-derived aldehyde may be prepared by periodate-mediated oxidation of a derivative of a protein, in which one or several of the available aspartic or glutamic acid residues has been used to acylate an amine of the general formula H₂N—R¹⁰—CH(XH)—CHR²⁰—WH, wherein R¹⁰ represents an organic diradical, R²⁰ represents an organic radical, and each W independently represents O or NH. Such an acylation may be accomplished selectively by treating a protein with an excess of said amine and a suitable enzyme, such as a glutamyl or aspartyl transpeptidase.

Alternatively, a protein-derived aldehyde of ketone may be prepared by coupling a thiol of the general formula HS—R³⁰—C(═O)—R⁴⁰, wherein R³⁰ represents an organic diradical, and R⁴⁰ represents hydrogen or an organic radical, to one of the available tyrosine residues by means of a tyrosinase, e.g. a mushroom tyrosinase, as described in the literature (S. Ito et al., J. Med. Chem. 1981, 24, 673-677).

Alternatively, a protein-derived aldehyde of ketone may be prepared by coupling a thiol of the general formula HS—R⁵⁰—CH(WH)—CHR⁶⁰—WH, wherein R⁵⁰ represents an organic diradical, R⁶⁰ represents hydrogen or an organic radical, and each W independently represents O or NH, followed by periodate-mediated oxidation of the resulting product.

Alternatively, a protein-derived aldehyde or ketone may be prepared by amide formation of the carboxy-terminal of said protein with an unnatural α-amino acid amide, which contains a ketone or an aldehyde as side-chain functional group. Such an unnatural α-amino acid amide may be coupled with said protein with the aid of an enzyme, such as a carboxypeptidase.

Alternatively, transglutaminase may be used to introduce a moiety in a protein comprising glutamine or lysine, and in particular glutamine. Said moiety may comprise a aldehyde or ketone, or it may comprise a latent aldehyde or ketone which upon further reaction, e.g. oxidation, is transformed to an aldehyde or a ketone.

The above discussed reaction are highly suitable means for attaching PEG to proteins with a pI with is approximately 2 units than the pH at which the reaction is run. In particular, the reactions are suitable for PEGylating acid proteins, i.e. proteins with pI below 7, such as below 6. Particular examples of acidic proteins include gastrin, glucagons, IL-10 β-chain receptor, IL-20 β-chain receptor, INFα, IL-18, members of the IL-1 family, members of the IL-9 family, members of the IL-10 family, IL-32, interferon regulatory factor 1, integrin α-IIb, melanoma associated antigen 1, ADP-sugar pyrophosphatase, orexigenic neuropeptide, tubulin-specific chaperone A, trefoil factor 2, trefoil factor 3, prothrombin, lymphotoxin-beta, tenomodulin, T-lymphokine-activated killer cell-originated protein kinase, vitronectin, insulin, growth hormone (GH), and in particular human growth hormone (hGH).

In one embodiment, GH is human growth hormone which has an amino acid sequence as set forth in SEQ ID No:1.

In one embodiment, GH is a variant of hGH, wherein a variant is understood to be the compound obtained by substituting one or more amino acid residues in the hGH sequence with another natural or unnatural amino acid; and/or by adding one or more natural or unnatural amino acids to the hGH sequence; and/or by deleting one or more amino acid residue from the hGH sequence, wherein any of these steps may optionally be followed by further derivatization of one or more amino acid residue. In particular, such substitutions are conservative in the sense that one amino acid residue is substituted by another amino acid residue from the same group, i.e. by another amino acid residue with similar properties. Amino acids may conveniently be divided in the following groups based on their properties: Basic amino acids (such as arginine and lysine), acidic amino acids (such as glutamic acid and aspartic acid), polar amino acids (such as glutamine, histidine, cysteine and asparagine), hydrophobic amino acids (such as leucine, isoleucine, proline, methionine and valine), aromatic amino acids (such as phenylalanine, tryptophan, tyrosine) and small amino acids (such as glycine, alanine, serine and threonine.).

In one embodiment, GH has at least 80%, such as at least 85%, such as at least 90%, such as at least 95% identity with hGH. In one embodiment, said identities to hGH is coupled to at least 20%, such as at least 40%, such as at least 60%, such as at least 80% of the growth hormone activity of hGH as determined in assay I herein.

The term “identity” as known in the art, refers to a relationship between the sequences of two or more proteins, as determined by comparing the sequences. In the art, “identity” also means the degree of sequence relatedness between proteins, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related proteins can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two proteins for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci. USA, 89:10915-10919 (1992) for the BLOSUM 62 comparison matrix) is also used by the algorithm.

Preferred parameters for a protein sequence comparison include the following:

Algorithm: Needleman et al., J. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for protein comparisons (along with no penalty for end gaps) using the GAP algorithm.

In one embodiment, GH is hGH extended with up to 100 amino acid residues at the N-terminal. In particular, said extension is up to 50, such as up to 40, such as up to 20, such as up to 10, such as up to 5, such as 1, 2 or 3 amino acid residues. A particular example of a GH variant is Ser-hGH or Ser-Xₙ-hGH, wherein Xₙ represents 1, 2, 3 or 4 natural or unnatural amino acids.

It should be clear from the above discussion of the invention, that the GH variant should maintain its acidity, i.e. it should have a pI below 7, such as below 6. Moreover, if the GH variant does not comprise an aldehyde or a ketone, it must be derivatised as discussed above to comprise one of these functionalities.

In one embodiment, the invention relates to PEGylated proteins obtained by the reaction of the present invention. In particular, the invention relates to a compound of formula II

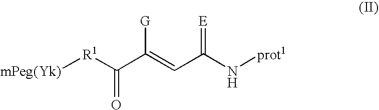

wherein Y is an integer from 1 to 140;
R¹ represents a diradical selected from

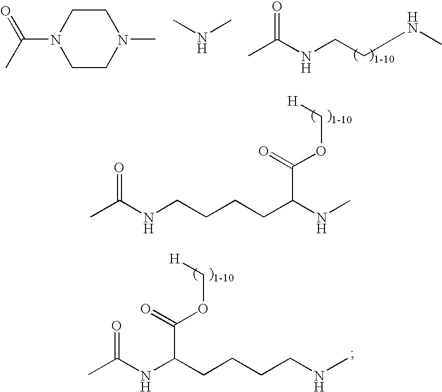

G represents an electron-withdrawing group or hydrogen;
E represents O or H₂;
and -prot¹ represents a radical derived from a protein by formal removal of the N-terminal amino group, or of the hydroxyl group of a glutamic acid side chain.

In one embodiment, Z represents a group selected from cyano, nitro, P(R²)₃⁺X⁻, —S(═O)R³, —S(═O)₂R⁴, or —C(═O)R⁵, wherein X represents halogen, BF₄, or PF₆;

R², R³, R⁴, and R⁵ independently represent aryl, optionally substituted with halogen, C₁₋₆-alkyl, cyano, or carboxyl; C₁₋₆-alkyl, optionally substituted with cyano; or NR⁶R⁷, wherein R⁶ and R⁷ independently represent hydrogen or C₁₋₆-alkyl. In particular, Z represents cyano or —PPh₃Cl, wherein Ph represent phenyl. In one embodiment, Z represents hydrogen. In one embodiment, E represents O. In one embodiment, Y represents 20, 40 or 60.

In one embodiment, the invention relates to a compound of formula III

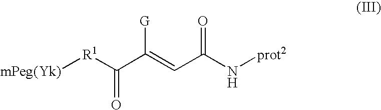

wherein Y, R¹, and G are defined as discussed above for formula II, and -prot² represents a radical derived from a protein by formal removal of the N-terminal amino group.

In one embodiment, the invention relates to a compound of formula IV

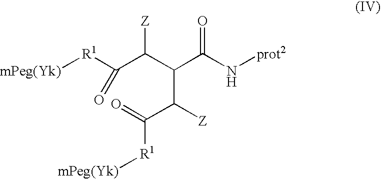

wherein Y is an integer from 1 to 140;
R¹ represents a diradical selected from

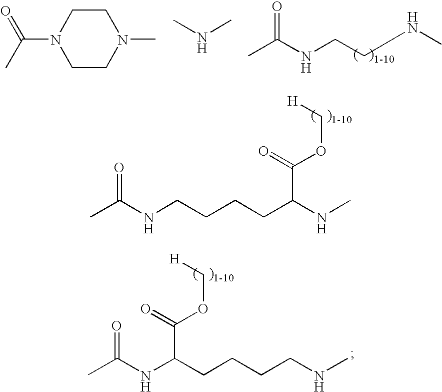

Z represents an electron-withdrawing group; and
prot² represents a radical derived from a protein by formal removal of the N-terminal amino group. In particular, Z represent a group selected from cyano, nitro, P(R²)₃⁺X⁻, —S(═O)R³, —S(═O)₂R⁴, or —C(═O)R⁵, wherein X represents halogen, BF₄, or PF₆;
R², R³, R⁴, and R⁵ independently represent aryl, optionally substituted with halogen, C₁₋₆-alkyl, cyano, or carboxyl; C₁₋₆-alkyl, optionally substituted with cyano; or NR⁶R⁷, wherein R⁶ and R⁷ independently represent hydrogen or C₁₋₆-alkyl. In particular, Z represents cyano or —PPh₃Cl, wherein Ph represent phenyl.

Particular examples of prot¹ and prot² include GH such as hGH or Ser-hGH radicals obtained by the formal removal of the N-terminal amino group.

Particular examples of compounds of the present invention include

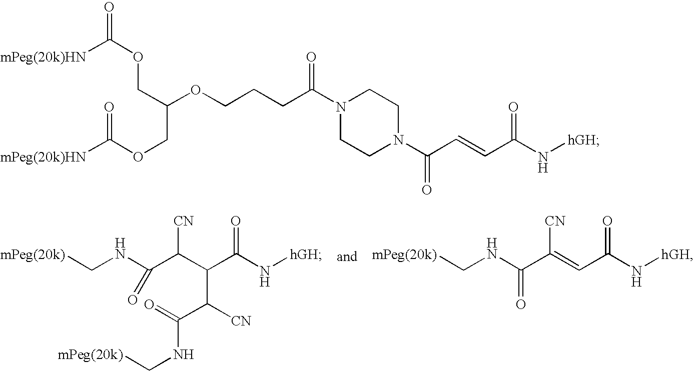

wherein hGH represent the radical obtained by formal removal of the N-terminal amino group from hGH. The systematic names of the above compounds are
N^{α1}-(4-(4-(4-(1,3-bis(mPEG(20K)oxy)-2-propyloxy)butyryl)piperazin-1-yl)fumar-1-yl)-hGH;
N^{α1}-(4-(mPEG(20k)ylamino)-3-cyanofumar-1-yl)-hGH; and
N^{α1}-(2,2-bis(1-(mPEG(20k)ylaminocarbonyl)-1-cyanomethyl)acetyl-hGH, respectively.

The compounds of formula II, III and IV, and in particular compounds according to formula II, III, and IV comprising a GH may have improved or alternative pharmacological properties compared to the corresponding un-conjugated GH, also referred to as the parent GH. Examples of such pharmacological properties include functional in vivo half-life, immunogenicity, renal filtration protease protection and albumin binding.

The term “functional in vivo half-life” is used in its normal meaning, i.e., the time at which 50% of the biological activity of the GH or conjugated GH is still present in the body/target organ, or the time at which the activity of the GH or GH conjugate is 50% of its initial value. As an alternative to determining functional in vivo half-life, “in vivo plasma half-life” may be determined, i.e., the time at which 50% of the GH or GH conjugate circulate in the plasma or bloodstream prior to being cleared. Determination of plasma half-life is often more simple than determining functional half-life and the magnitude of plasma half-life is usually a good indication of the magnitude of functional in vivo half-life. Alternative terms to plasma half-life include serum half-life, circulating half-life, circulatory half-life, serum clearance, plasma clearance, and clearance half-life.

The term “increased” as used in connection with the functional in vivo half-life or plasma half-life is used to indicate that the relevant half-life of the GH conjugate is statistically significantly increased relative to that of the parent GH, as determined under comparable conditions. For instance the relevant half-life may be increased by at least about 25%, such as by at lest about 50%, e.g., by at least about 100%, 150%, 200%, 250%, or 500%. In one embodiment, the compounds of the present invention exhibit an increase in half-life of at least about 5 h, preferably at least about 24 h, more preferably at least about 72 h, and most preferably at least about 7 days, relative to the half-life of the parent GH.

Measurement of in vivo plasma half-life can be carried out in a number of ways as described in the literature. An increase in in vivo plasma half-life may be quantified as a decrease in clearance (CL) or as an increase in mean residence time (MRT). Conjugated GH of the present invention for which the CL is decreased to less than 70%, such as less than 50%, such than less than 20%, such than less than 10% of the CL of the parent GH as determined in a suitable assay is said to have an increased in vivo plasma half-life. Conjugated GH of the present invention for which MRT is increased to more than 130%, such as more than 150%, such as more than 200%, such as more than 500% of the MRT of the parent GH in a suitable assay is said to have an increased in vivo plasma half-life. Clearance and mean residence time can be assessed in standard pharmacokinetic studies using suitable test animals. It is within the capabilities of a person skilled in the art to choose a suitable test animal for a given protein. Tests in human, of course, represent the ultimate test. Suitable text animals include normal, Sprague-Dawley male rats, mice and cynomolgus monkeys. Typically the mice and rats are in injected in a single subcutaneous bolus, while monkeys may be injected in a single subcutaneous bolus or in a single iv dose. The amount injected depends on the test animal. Subsequently, blood samples are taken over a period of one to five days as appropriate for the assessment of CL and MRT. The blood samples are conveniently analysed by ELISA techniques.

The term “Immunogenicity” of a compound refers to the ability of the compound, when administered to a human, to elicit a deleterious immune response, whether humoral, cellular, or both. In any human sub-population, there may exist individuals who exhibit sensitivity to particular administered proteins. Immunogenicity may be measured by quantifying the presence of growth hormone antibodies and/or growth hormone responsive T-cells in a sensitive individual, using conventional methods known in the art. In one embodiment, the conjugated GH of the present invention exhibit a decrease in immunogenicity in a sensitive individual of at least about 10%, preferably at least about 25%, more preferably at least about 40% and most preferably at least about 50%, relative to the immunogenicity for that individual of the parent GH. In another aspect, immunogenicity may refer to the typical response in a population of similar subjects, such as the typical response in a patient population in a clinical trial.

The term “protease protection” or “protease protected” as used herein is intended to indicate that the conjugated GH of the present invention is more resistant to the plasma peptidase or proteases than is the parent GH. Protease and peptidase enzymes present in plasma are known to be involved in the degradation of circulating proteins, such as e.g. circulating peptide hormones, such as growth hormone.

Resistance of a protein to degradation by for instance dipeptidyl aminopeptidase IV (DPPIV) is determined by the following degradation assay: Aliquots of the protein (5 nmol) are incubated at 37° C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the protein degradation products are separated and quantified using HPLC analysis. One method for performing this analysis is: The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999; 79:93-102 and Mentlein et al. Eur. J. Biochem. 1993; 214:829-35. Proteins and their degradation products may be monitored by their absorbance at 220 nm (peptide bonds) or 280 nm (aromatic amino acids), and are quantified by integration of their peak areas related to those of standards. The rate of hydrolysis of a protein by dipeptidyl aminopeptidase IV is estimated at incubation times which result in less than 10% of the peptide being hydrolysed. The resistance to other plasma proteases or peptidases may be determined in similar ways. In one embodiment, the rate of hydrolysis of the GH conjugate is less than 70%, such as less than 40%, such as less than 10% of that of the parent GH.

The most abundant protein component in circulating blood of mammalian species is serum albumin, which is normally present at a concentration of approximately 3 to 4.5 grams per 100 mL of whole blood. Serum albumin is a blood protein of approximately 70,000 daltons which has several important functions in the circulatory system. It functions as a transporter of a variety of organic molecules found in the blood, as the main transporter of various metabolites such as fatty acids and bilirubin through the blood, and, owing to its abundance, as an osmotic regulator of the circulating blood. Serum albumin has a half-life of more than one week, and one approach to increasing the plasma half-life of proteins has been to conjugate to the protein a group that binds to serum albumin. Albumin binding property may be determined as described in J. Med. Chem., 43, 2000, 1986-1992, which is incorporated herein by reference.

Compounds of formula II, III and IV when said compounds comprise GH exert growth hormone activity and may as such be used in the treatment of diseases or states which will benefit from an increase in the amount of circulating growth hormone. In particular, the invention provides a method for the treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucucorticoid treatment in children, the method comprising administering to a patient in need thereof a therapeutically effective amount of a compound according to formula II, III or IV, wherein said compound comprises GH.

In one aspect, the invention provides a method for the acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a therapeutically effective amount of a compound of formula II, III or IV, wherein said compound comprises GH.

In one embodiment, the invention relates to the use of compounds according to formula II, III or IV, wherein said compound comprises GH in the manufacture of diseases benefiting from an increase in the growth hormone plasma level, such as the disease mentioned above.

A typical parenteral dose is in the range of 10⁻⁹ mg/kg to about 100 mg/kg body weight per administration. Typical administration doses are from about 0.0000001 to about 10 mg/kg body weight per administration. The exact dose will depend on e.g. indication, medicament, frequency and mode of administration, the sex, age and general condition of the subject to be treated, the nature and the severity of the disease or condition to be treated, the desired effect of the treatment and other factors evident to the person skilled in the art.

Typical dosing frequencies are twice daily, once daily, bi-daily, twice weekly, once weekly or with even longer dosing intervals. Due to the prolonged half-lifes of the fusion proteins of the present invention, a dosing regime with long dosing intervals, such as twice weekly, once weekly or with even longer dosing intervals is a particular embodiment of the invention.

Many diseases are treated using more than one medicament in the treatment, either concomitantly administered or sequentially administered. It is therefore within the scope of the present invention to use compounds of formula II, III or IV, wherein said compound comprises GH in therapeutic methods for the treatment of one of the above mentioned diseases in combination with one or more other therapeutically active compound normally used in the treatment said diseases. By analogy, it is also within the scope of the present invention to use compounds of formula II, III or IV, wherein said compound comprises GH in combination with other therapeutically active compounds normally used in the treatment of one of the above mentioned diseases in the manufacture of a medicament for said disease.

### Pharmaceutical Compositions

Another purpose is to provide a pharmaceutical composition comprising a conjugated GH of the present invention which is present in a concentration from 10-15 mg/ml to 200 mg/ml, such as e.g. 10-10 mg/ml to 5 mg/ml and wherein said composition has a pH from 2.0 to 10.0. The composition may further comprise a buffer system, preservative(s), tonicity agent(s), chelating agent(s), stabilizers and surfactants. In one embodiment of the invention the pharmaceutical composition is an aqueous composition, i.e. composition comprising water. Such composition is typically a solution or a suspension. In a further embodiment of the invention the pharmaceutical composition is an aqueous solution. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical composition is a freeze-dried composition, whereto the physician or the patient adds solvents and/or diluents prior to use.

In another embodiment the pharmaceutical composition is a dried composition (e.g. freeze-dried or spray-dried) ready for use without any prior dissolution.

In a further aspect the invention relates to a pharmaceutical composition comprising an aqueous solution of a GH conjugate, and a buffer, wherein said GH conjugate is present in a concentration from 0.1-100 mg/ml or above, and wherein said composition has a pH from about 2.0 to about 10.0.

In a another embodiment of the invention the pH of the composition is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the composition further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In a further embodiment of the invention the composition further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C₄-C₈ hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects obtained using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

In a further embodiment of the invention the composition further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 20^{th} edition, 2000.

In a further embodiment of the invention the composition further comprises a stabilizer. The use of a stabilizer in pharmaceutical compositions is well-known to the skilled person. For convenience reference is made to Remington: The Science and Practice of Pharmacy, 20^{th} edition, 2000.

More particularly, compositions of the invention are stabilized liquid pharmaceutical compositions whose therapeutically active components include a protein that possibly exhibits aggregate formation during storage in liquid pharmaceutical compositions. By “aggregate formation” is intended a physical interaction between the protein molecules that results in formation of oligomers, which may remain soluble, or large visible aggregates that precipitate from the solution. By “during storage” is intended a liquid pharmaceutical composition or composition once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or composition is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a protein during storage of a liquid pharmaceutical composition can adversely affect biological activity of that protein, resulting in loss of therapeutic efficacy of the pharmaceutical composition. Furthermore, aggregate formation may cause other problems such as blockage of tubing, membranes, or pumps when the protein-containing pharmaceutical composition is administered using an infusion system.

The pharmaceutical compositions of the invention may further comprise an amount of an amino acid base sufficient to decrease aggregate formation by the protein during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L or D isomer, or mixtures thereof) of a particular amino acid (methionine, histidine, arginine, lysine, isoleucine, aspartic acid, tryptophan, threonine and mixtures thereof) or combinations of these stereoisomers or glycine or an organic base such as but not limited to imidazole, may be present in the pharmaceutical compositions of the invention so long as the particular amino acid or organic base is present either in its free base form or its salt form. In one embodiment the L-stereoisomer of an amino acid is used. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the protein during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the protein acting as the therapeutic agent is a protein comprising at least one methionine residue susceptible to such oxidation. By “inhibit” is intended minimal accumulation of methionine oxidized species over time. Inhibiting methionine oxidation results in greater retention of the protein in its proper molecular form. Any stereoisomer of methionine (L or D isomer) or any combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be obtained by adding methionine such that the ratio of methionine added to methionine residues ranges from about 1:1 to about 1000:1, such as 10:1 to about 100:1.

In a further embodiment of the invention the composition further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active protein therein. Stabilizing agents of particular interest to the present invention include, but are not limited to, methionine and EDTA, which protect the protein against methionine oxidation, and a nonionic surfactant, which protects the protein against aggregation associated with freeze-thawing or mechanical shearing.

In a further embodiment of the invention the composition further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C₆-C₁₂ (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 20^{th} edition, 2000.

It is possible that other ingredients may be present in the pharmaceutical composition of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical composition of the present invention.

Pharmaceutical compositions containing a GH conjugate according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the GH conjugate, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the composition of solids, semisolids, powder and solutions for pulmonary administration of GH conjugate, using, for example a metered dose inhaler, dry powder inhaler and a nebulizer, all being devices well known to those skilled in the art.

Compositions of the current invention are specifically useful in the composition of controlled, sustained, protracting, retarded, and slow release drug delivery systems. More specifically, but not limited to, compositions are useful in composition of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the GH conjugate in the form of a nasal or pulmonal spray. As a still further option, the pharmaceutical compositions containing the GH conjugate of the invention can also be adapted to transdermal administration, e.g. by needle-free injection or from a patch, optionally an iontophoretic patch, or transmucosal, e.g. buccal, administration.

The term “stabilized composition” refers to a composition with increased physical stability, increased chemical stability or increased physical and chemical stability.

The term “physical stability” of the protein composition as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein compositions is evaluated by means of visual inspection and/or turbidity measurements after exposing the composition filled in suitable containers (e.g. cartridges or vials) to mechanical/physical stress (e.g. agitation) at different temperatures for various time periods. Visual inspection of the compositions is performed in a sharp focused light with a dark background. The turbidity of the composition is characterized by a visual score ranking the degree of turbidity for instance on a scale from 0 to 3 (a composition showing no turbidity corresponds to a visual score 0, and a composition showing visual turbidity in daylight corresponds to visual score 3). A composition is classified physical unstable with respect to protein aggregation, when it shows visual turbidity in daylight. Alternatively, the turbidity of the composition can be evaluated by simple turbidity measurements well-known to the skilled person. Physical stability of the aqueous protein compositions can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein composition as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein composition can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized composition” refers to a composition with increased physical stability, increased chemical stability or increased physical and chemical stability. In general, a composition must be stable during use and storage (in compliance with recommended use and storage conditions) until the expiration date is reached.

In one embodiment of the invention the pharmaceutical composition comprising the GH conjugate is stable for more than 6 weeks of usage and for more than 3 years of storage.

In another embodiment of the invention the pharmaceutical composition comprising the GH conjugate is stable for more than 4 weeks of usage and for more than 3 years of storage.

In a further embodiment of the invention the pharmaceutical composition comprising the GH conjugate is stable for more than 4 weeks of usage and for more than two years of storage.

In an even further embodiment of the invention the pharmaceutical composition comprising the GH conjugate is stable for more than 2 weeks of usage and for more than two years of storage.

### EXAMPLES

In the examples the following terms are intended to have the following, general meanings:

Boc: tert-butyloxycarbonyl
Bt: 1-benzotriazolyl
DABCO: 1,4-diazabicyclo[2.2.2]octane
DBU: 1,8-diazabicyclo[5.4.0]undec-7-ene
DCM: dichloromethane, methylenechloride
DIC: diisopropylcarbodiimide
DIPEA: diisopropylethylamine

### DMA: N,N-dimethylacetamide

DMF: N,N-dimethyl formamide
DMSO: dimethyl sulfoxide
DMAP: 4-dimethylaminopyridine
DMPU: 1,3-dimethyltetrahydropyrimidin-2-one
EDC or EDAC: N-ethyl-N′-(3-dimethylaminopropyl)carbodiimide hydrochloride
Fmoc: 9-fluorenylmethyloxycarbonyl
HBTU: 2-(1H-Benzotriazol-1-yl-)-1,1,3,3 tetramethyluronium hexafluorophosphate
HOAt: 3-hydroxy-3H-[1,2,3]triazolo[4,5-b]pyridine, 4-aza-3-hydroxybenzotriazole
HOBt: N-hydroxybenzotriazole, 1-hydroxybenzotriazole

### HONSu: N-hydroxysuccinimide

### NMP: N-methylpyrrolidone

HPLC: high pressure liquid chromatography
Pmc 2,2,5,7,8-pentamethylchroman-6-sulfonyl r.t. room temperature
Su: succinimidyl
TFA trifluoroacetic acid
TIS triisopropylsilane
Trt: trityl, triphenylmethyl
Ts: toluenesulfonyl
TSTU O-(1-succinimidyl)-N,N,N′,N′-tetramethyluronium hexafluorophosphate

NMR spectra were recorded on Bruker 300 MHz and 400 MHz instruments. HPLC-MS was performed on a Perkin Elmer instrument (API 100).

HPLC-systems from Merck-Hitachi (Hibar™ RT 250-4, Lichrosorb™ RP 18, 5.0 μm, 4.0×250 mm, gradient elution, 20% to 80% acetonitrile in water within 30 min, 1.0 ml/min, detection at 254 nm) and Waters (Symmetry™, C18, 3.5 μm, 3.0×150 mm, gradient elution, 5% to 90% acetonitrile in water within 15 min, 1.0 ml/min, detection at 214 nm) were used.

The reverse phase analysis was performed using UV detections at 214, 254, 276 and 301 nm on a 218TP54 4.6 mm×150 mm C-18 silica column, which was eluted at 1 ml/min at 42° C. The column was equilibrated with 5% acetonitrile, 85% water and 10% of a solution of 0.5% trifluoroacetic acid in water and eluted by a linear gradient from 5% acetonitrile, 85% water and 10% of a solution of 0.5% trifluoroacetic acid to 90% acetonitrile and 10% of a solution of 0.5% trifluoroacetic acid over 15 min.

Furthermore, where stated the following HPLC method A was used:

The RP-analysis was performed using a Waters 2690 systems fitted with a Waters 996 diode array detector. UV detections were collected at 214, 254, 276, and 301 nm on a 218TP54 4.6 mm×250 mm 5μ C-18 silica column (The Seperations Group, Hesperia), which was eluted at 1 ml/min at 42° C. The column was equilibrated with 5% acetonitrile (+0.1% TFA) in an aqueous solution of TFA in water (0.1%). After injection, the sample was eluted by a gradient of 0% to 90% acetonitrile (+0.1% TFA) in an aqueous solution of TFA in water (0.1%) during 50 min.

PEGylated growth hormone was purified by ion-exchange chromatography in the following way:

### Column: Mono Q HR 10/10

Apparatus: ÄKTA purifier

### Buffer A: 10 mM Tris, pH 8.0

### Buffer B: 10 mM Tris, 200 mM NaCl pH 8.0

The column is equilibrated with 5 column volumes (CV)
Flow: 4.0 ml/min

### Detector: 280 nm

Fraction size: 2 ml

### Washing: 2 CV

Gradient: 0% B to 100% B over 20 CV

### Example 1

### mPEG(40k)-hGH by Wittig Reaction

A. Preparation of the mPEG(40k)-Derived Phosphonium Salt

### (1-(4-(4-(1,3-bis(mPEG(20K)oxy)-2-propyloxy)butyryl)piperazin-1-yl)acet-2-yl)triphenylphosphonium chloride

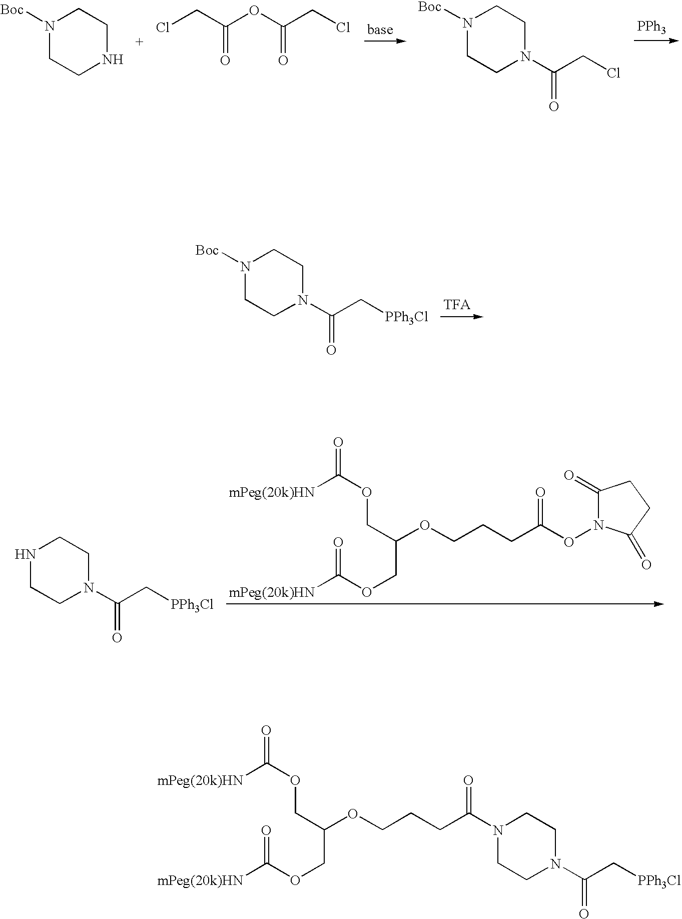

To a solution of Boc-piperazine (0.63 g, 3.38 mmol) in DCM (20 ml) were added DIPEA (1.0 ml, 5.80 mmol) and then, in one portion, chloroacetic anhydride (0.52 g, 3.04 mmol). The mixture was stirred at room temperature for one hour. Water (50 ml) and 1N HCl (20 ml) were added, and the product was extracted with DCM (3×). The combined extracts were washed with brine, dried over MgSO₄, and concentrated under reduced pressure. 0.76 g (95%) of 1-Boc-4-chloroacetylpiperazine was obtained as an oil which slowly crystallized.

¹H NMR (d₆-DMSO) δ 1.41 (s, 9H), 3.28-3.47 (m, 8H), 4.39 (s, 2H).

Conversion to phosphonium salt: A mixture of 1-Boc-4-chloroacetylpiperazine (0.76 g, 2.89 mmol), toluene (10 ml), and triphenylphosphine (1.5 g, 5.72 mmol) was stirred at 90° C. After 1.5 h a thick precipitate had formed and more toluene (5 ml) was added. Heating was interrupted after a total of 4 h. Filtration, washing of the solid with toluene, and drying under reduced pressure yielded 0.81 g (53%) of (1-(4-(tert-butyloxycarbonyl)piperazin-1-yl)acet-2-yl)triphenylphosphonium chloride as a solid.

¹H NMR (d₆-DMSO) δ 1.41 (s, 9H), 3.22-3.45 (m, 6H), 3.60 (m, 2H), 5.54 (d, J=13.8 Hz, 2H), 7.69-7.88 (m, 15H).

Deprotection and acylation: To (1-(4-(tert-butyloxycarbonyl)piperazin-1-yl)acet-2-yl)triphenylphosphonium chloride (170 mg, 0.32 mmol) were added DCM (10 ml) and TFA (10 ml). After 0.5 h the mixture was concentrated, and the residue coevaporated once with a mixture of acetonitrile and toluene. The residue was redissolved in DCM (7.5 ml), DIPEA (1.0 ml) was added, and the resulting solution was added to mPEG(40k)-NHS (1.0 g, 0.025 mmol). The mixture was stirred at room temperature for 5 d. The product was purified by fivefold precipitation with Et₂O (100-200 ml) and redissolution in DCM. Drying under reduced pressure yielded 1.08 g of the title compound.

¹H NMR (d₆-DMSO) δ 3-4 (m), 5.44 (d, J=14 Hz), 7.65-7.90 (m).

B: Oxidation and Wittig reaction of Ser-hGH

### N^{α1}-(4-(4-(4-(1,3-bis(mPEG(20K)oxy)-2-propyloxy)butyryl)piperazin-1-yl)fumar-1-yl)-hGH

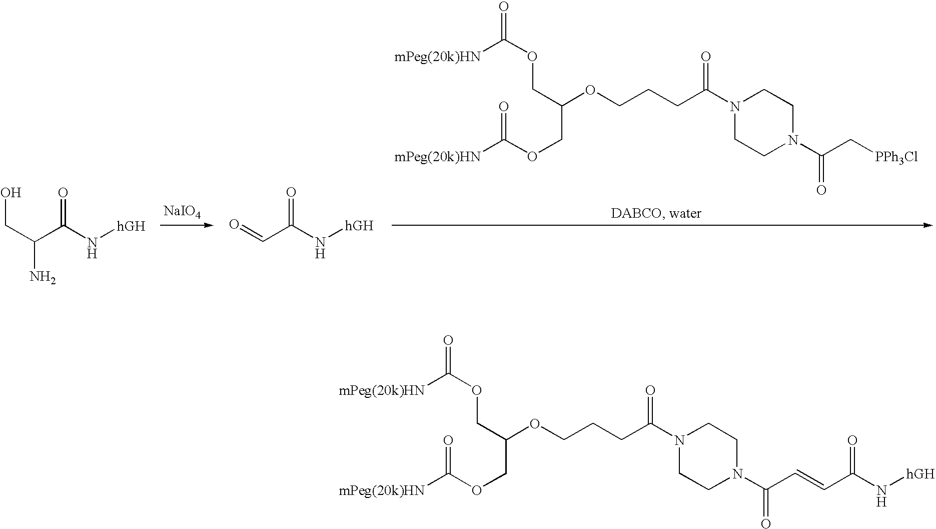

The following solutions were prepared:

Buffer A: 135 mg triethanolamine and 290 mg 3-methylthio-1-propanol in water (20 ml)
Buffer B: 3-methylthio-1-propanol (1.2 g) in water (80 ml)
Sodium periodate solution: NaIO₄: 48.2 mg in 1.0 ml water

The Peg(40k)-phosphonium salt prepared as described above (19 mg, 475 nmol) and DABCO (20 mg) were dissolved in buffer B (0.40 ml).

SerhGH (20 mg, 900 nmol) was dissolved in buffer A (4 ml), and the periodate solution (0.40 ml) was added. After 15 min the mixture was diluted with buffer B and dialized 4 times with this buffer (Millipore, Amicon, cut-off 5000, 20-30 min). The residue (0.3 ml) was transferred to a vial, and the tube was rinsed with buffer B (0.1 ml). The resulting solution (0.4 ml) was added to the solution of the phosphonium salt. After standing for 18 h at room temperature, analysis indicated 30% yield of the title compound.

### Example 2

### mPEG(40k)-hGH and mPEG(20k)-hGH by Knoevenagel and Michael Reaction

A. Preparation of the mPEG(20k)-Derived Cyanoacetamide

### N-(mPEG(20k)yl)cyanoacetamide

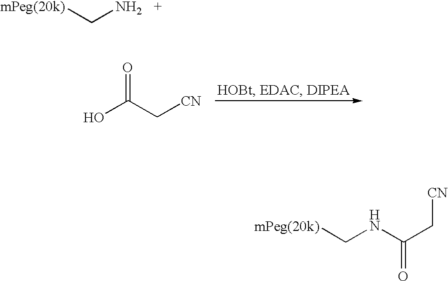

To a solution of Peg(20k)-NH₂ (1.0 g, 0.05 mmol) in DCM (10 ml) were added cyanoacetic acid (57 mg, 0.67 mmol), HOBt (85 mg, 0.63 mmol), EDAC (112 mg, 0.58 mmol), and then dropwise DIPEA (0.18 ml, 1.04 mmol). After stirring at room temperature for 24 h the mixture was poured into Et₂O (100 ml), stirred, filtered, and redissolved in DCM (10 ml). The precipitation/redissolution was repeated 5 times.

B: Oxidation and Knoevenagel-Michael reaction of Ser-hGH

### N^{α1}-(4-(mPEG(20k)ylamino)-3-cyanofumar-1-yl)-hGH and Na^{α1}-(2,2-bis(1-(mPEG(20k)ylaminocarbonyl)-1-cyanomethyl)acetyl-hGH

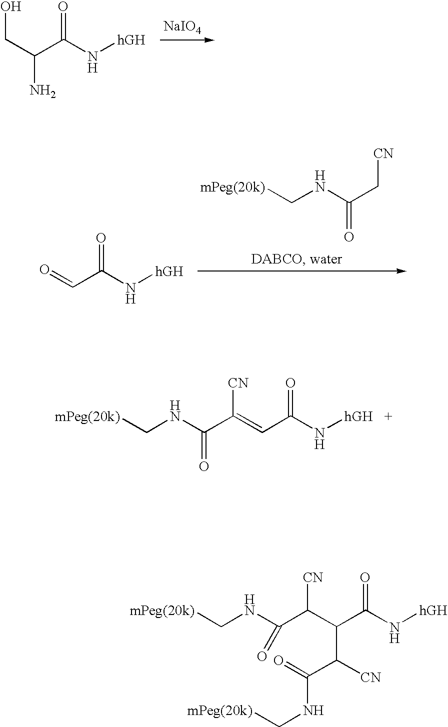

The Peg(20k)-cyanoacetamide (10 mg, 500 nmol) and DABCO (20 mg) were dissolved in buffer B (0.40 ml).

SerhGH (20 mg, 900 nmol) was dissolved in buffer A (4 ml), and the periodate solution (0.40 ml) was added. After 15 min the mixture was diluted with buffer B and dialized 4 times with this buffer (Millipore, Amicon, cut-off 5000, 20-30 min). The residue (0.3 ml) was transferred to a vial, and the tube was rinsed with buffer B (0.1 ml). The resulting solution (0.4 ml) was added to the solution of the cyanoacetamide. After standing for 18 h at room temperature, analysis indicated the mixture to contain an equimolar mixture of N^{α1}-glyoxylyl-hGH, N^{α1}-(4-(mPEG(20k)ylamino)-3-cyanofumar-1-yl)-hGH, and N^{α1}-(2,2-bis(1-(mPEG(20k)ylaminocarbonyl)-1-cyanomethyl)acetyl-hGH.

### Pharmacological Methods

### Assay (I) BAF-3 GHR Assay to Determine Growth Hormone Activity

The BAF-3 cells (a murine pro-B lymphoid cell line derived from the bone marrow) was originally IL-3 dependent for growth and survival. II-3 activates JAK-2 and STAT which are the same mediators GH is activating upon stimulation. After transfection of the human growth hormone receptor the cell line was turn into a growth hormone-dependent cell line. This clone can be used to evaluate the effect of different growth hormone samples on the survival of the BAF-3 GHR.

The BAF-3 GHR cells are grown in starvation medium (culture medium without growth hormone) for 24 hours at 37° C., 5% CO₂.

The cells are washed and re-suspended in starvation medium and seeded in plates. 10 μl of growth hormone compound or human growth hormone in different concentrations or control is added to the cells, and the plates are incubated for 68 hours at 37° C., 5% CO₂.

AlamarBlue® is added to each well and the cells are then incubated for another 4 hours. The AlamarBlue® is a redox indicator, and is reduced by reactions innate to cellular metabolism and, therefore, provides an indirect measure of viable cell number.

Finally, the metabolic activity of the cells is measure in a fluorescence plate reader. The absorbance in the samples is expressed in % of cells not stimulated with growth hormone compound or control and from the concentration-response curves the activity (amount of a compound that stimulates the cells with 50%) can be calculated.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). Basic and novel properties with respect to such aspects of the invention are provided here (e.g., in the context of a GH-comprising compounds of the invention, such properties include exhibition of GH-like biological activity).

1. A compound of formula I 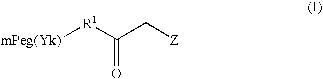
wherein
Y represents an integer from 1 to 140;
R¹ represents a diradical selected from 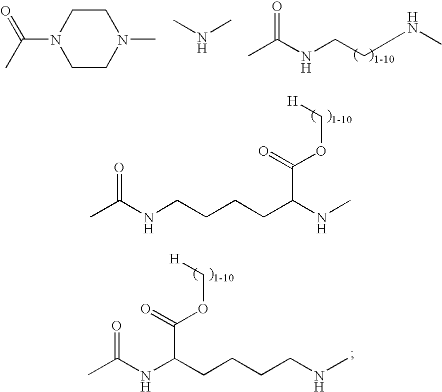
Z represents a group selected from cyano, nitro, —P(R²)₃⁺X⁻, —S(═O)R³, —S(═O)₂R⁴, or —C(═O)R⁵, wherein X represents halogen, BF₄, or PF₆;
R², R³, R⁴, and R⁵ independently represent aryl, optionally substituted with halogen, C₁₋₆-alkyl, cyano or carboxyl; C₁₋₆-alkyl, optionally substituted with cyano; or NR⁶R⁷,
wherein R⁶ and R⁷ independently represent hydrogen or C₁₋₆alkyl.

2. The compound according to claim 1, wherein Z represents cyano.

3. The compound according to claim 1, wherein Z represents —PPh₃Cl.

4. The compound according to claim 1, wherein R¹ represents

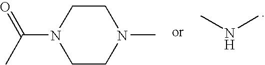

5. The compound according to claim 1, wherein Y represents 10, 20, 30, 40, or 60.

6. The compound according to claim 1 selected from

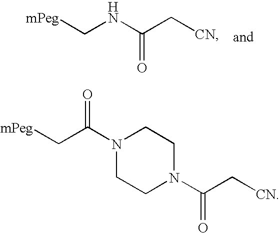

7. A method for the covalent attachment of PEG to proteins, the method comprising reacting a protein-derived aldehyde or ketone with a compound of formula I 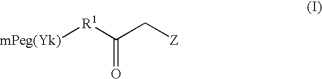
wherein
Y represents an integer from 1 to 140;
R¹ represents a diradical selected from 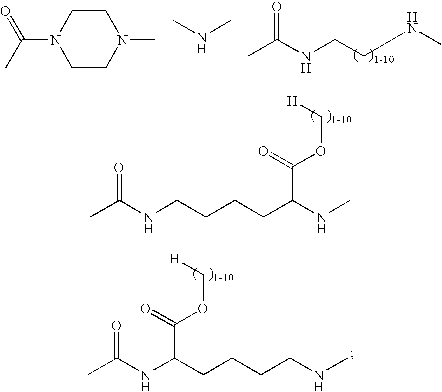
Z represents a group selected from cyano, nitro, —P(R²)₃⁺X⁻, —S(═O)R³, —S(═O)₂R⁴, or —C(═O)R⁵ wherein X represents halogen BF₄, or PF₆;
R², R³, R⁴, and R⁵ independently represent aryl optionally substituted with halogen C₁₋₆-alkyl cyano or carboxyl; C₁₋₆-alkyl, optionally substituted with cyano; or NR⁶R⁷,
wherein R⁶ and R⁷ independently represent hydrogen or C₁₋₆alkyl at pH>7.

8. The method according to claim 7, wherein the protein-derived aldehyde is N^{α1}-glyoxylyl-hGH.

9. The method according to claim 7, wherein the reaction is conducted in water.

10. The method according to claim 7, in which a tertiary amine is added as base to raise the pH.

11. The method as defined in claim 10, in which the tertiary amine is DABCO.

12. A compound of formula (II) 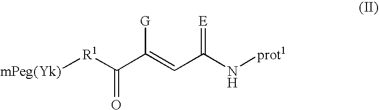
wherein Y represents an integer from 1 to 140;
R¹ represents a diradical selected from 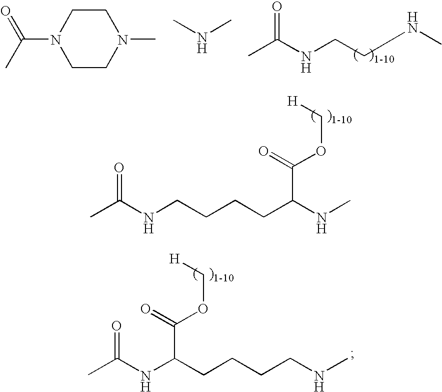
G represents hydrogen, cyano, nitro, —P(R²)₃⁺X⁻, —S(═O)R³, —S(═O)₂R⁴, or —C(═O)R⁵, wherein X represents halogen, BF₄, or PF₆;
R², R³, R⁴, and R⁵ independently represent aryl, optionally substituted with halogen, C₁₋₆-alkyl, cyano, or carboxyl; C₁₋₆-alkyl, optionally substituted with cyano; or NR⁶R⁷,
wherein R⁶ and R⁷ independently represent hydrogen or lower alkyl;
E represents O or H₂; and
prot¹ represents a radical derived from a protein by formal removal of the N-terminal amino group, or of the hydroxyl group of a glutamic acid side chain.

13. The compound according to claim 12, wherein Z represents cyano or hydrogen; E represents O; and Y represents 20, 40 or 60.

14. A compound of formula III 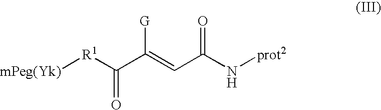
wherein Y represents an integer from 1 to 140;
R¹ represents a diradical selected from 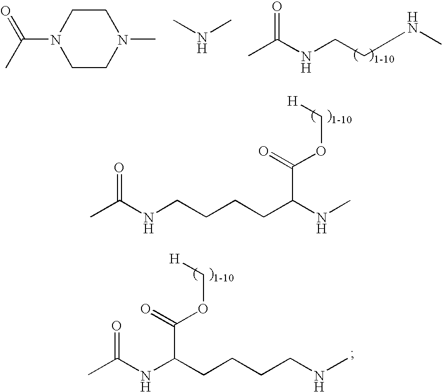
G represents hydrogen, cyano, nitro, —P(R²)₃⁺X⁻, —S(═O)R³, —S(═O)₂R⁴, or —C(═O)R⁵, wherein X represents halogen, BF₄, or PF₆;
R², R³, R⁴, and R⁵ independently represent aryl, optionally substituted with halogen, C₁₋₆-alkyl, cyano, or carboxyl; C₁₋₆-alkyl, optionally substituted with cyano; or NR⁶R⁷,
wherein R⁶ and R⁷ independently represent hydrogen or lower alkyl; and
prot² represents a radical derived from a protein by formal removal of the N-terminal amino group.

15. The compound according to claim 14, wherein Z represents cyano or hydrogen; E represents O, and Y represents 20, 40 or 60.

16. A compound according to formula IV 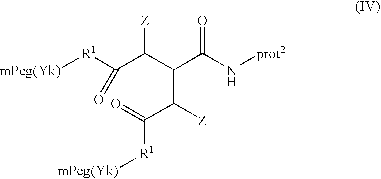
Y represents an integer from 1 to 140;
R¹ represents a diradical selected from 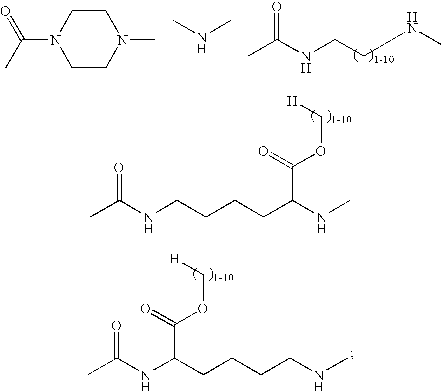
Z represents a group selected from cyano, nitro, —P(R²)₃⁺X⁻, —S(═O)R³, —S(═O)₂R⁴, or —C(═O)R⁵, wherein X represents halogen, BF₄, or PF₆;
, R³, R⁴, and R⁵ independently represent aryl, optionally substituted with halogen, C₁₋₆-alkyl, cyano, or carboxyl; C₁₋₆-alkyl, optionally substituted with cyano; or NR⁶R⁷,
wherein R⁶ and R⁷ independently represent hydrogen or C₁₋₆alkyl; and
prot² represents a radical derived from a protein by formal removal of the N-terminal amino group.

17. The compound according to claim 16, wherein Z represent cyano.

18. The compound according to claim 11, wherein -prot¹ or -prot² represents a growth hormone derived radical.

19. The compound according to claim 11, wherein -prot¹ or -prot² represents a growth hormone derived radical obtained by the formal removal of the N-terminal amino group.

20. The compound according to claim 18, wherein said radical is human growth hormone derived.

21. The compound according to claim 18, wherein said radical is human growth hormone derived radical obtained by the formal removal of the N-terminal amino group.

22. The compound according to claim 11 selected from

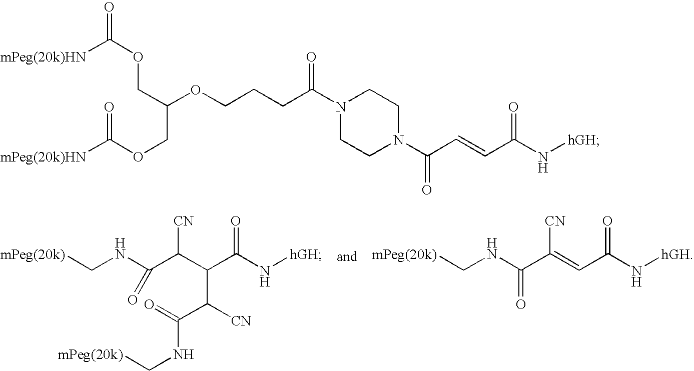

23. (canceled)

24. (canceled)

25. A pharmaceutical composition comprising a compound of formula I 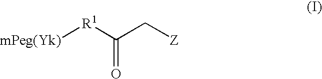
wherein
Y represents an integer from 1 to 140;
R¹ represents a diradical selected from 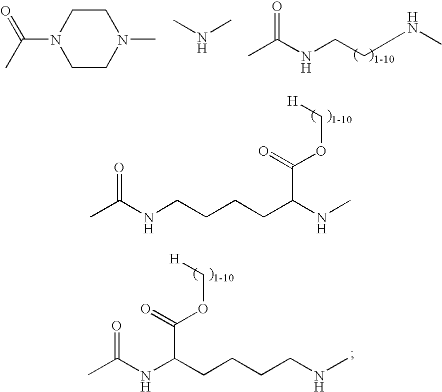
Z represents a group selected from cyano, nitro, —P(R²)₃⁺X⁻, —S(═O)R³, —S(═O)₂R⁴, or —C(═O)R⁵ wherein X represents halogen BF₄, or PF₆;
R², R³, R⁴, and R⁵ independently represent aryl optionally substituted with halogen C₁₋₆-alkyl cyano or carboxyl; C₁₋₆-alkyl, optionally substituted with cyano; or NR⁶R⁷,
wherein R⁶ and R⁷ independently represent hydrogen or C₁₋₆alkyl.

26. (canceled)

27. A method of treating a disease benefiting from an increase in the level of circulating growth hormone, the method comprising administering a therapeutically effective amount of a compound of formula I 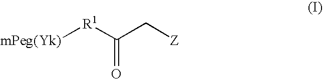
wherein
Y represents an integer from 1 to 140;
R¹ represents a diradical selected from 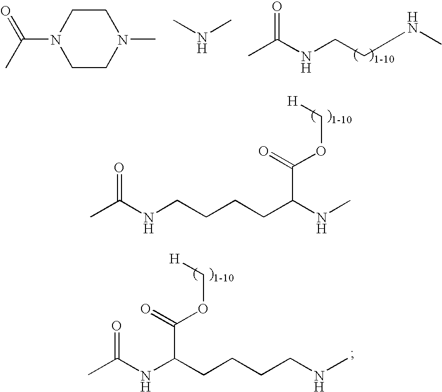
Z represents a group selected from cyano, nitro, —P(R²)₃⁺X⁻, —S(═O)R³—S(═O)₂R⁴, or —C(═O)R⁵ wherein X represents halogen BF₄ or PF₆;
R², R³, R⁴, and R⁵ independently represent aryl optionally substituted with halogen C₁₋₆-alkyl cyano or carboxyl; C₁₋₆-alkyl, optionally substituted with cyano; or NR⁶R⁷,
wherein R⁶ and R⁷ independently represent hydrogen or C₁₋₆alkyl to a patient in need thereof.

28. The method according to claim 27, wherein said administration takes place every second day or with longer intervals.

29. (canceled)

30. The compound according to claim 19, wherein said radical is human growth hormone derived.

31. The compound according to claim 19, wherein said radical is human growth hormone derived radical obtained by the formal removal of the N-terminal amino group
